(19) United States
(12) Patent Application Publication
(10) Pub. No.: US 2022/0151540 A1
(43) Pub. Date: May 19, 2022

(54) EXPLAINABLE ARTIFICIAL INTELLIGENCE SYSTEM FOR DIAGNOSIS OF MENTAL DISEASES AND THE CONTROL METHOD THEREOF

(71) Applicant: 4N Inc., Daejeon (KR)

(72) Inventors: Seongkyun KIM, Seoul (KR); Jaehyung KWON, Daejeon (KR); Hoon-Hee KIM, Daejeon (KR)

(73) Assignee: 4N Inc., Daejeon (KR)

(21) Appl. No.: 17/531,619

(22) Filed: Nov. 19, 2021

(30) Foreign Application Priority Data
Nov. 19, 2020 (KR) 10-2020-0155762
Nov. 20, 2020 (KR) 10-2020-0156656
Mar. 22, 2021 (KR) 10-2021-0036451
Mar. 22, 2021 (KR) 10-2021-0036452

(51) Int. Cl. A61B 5/00; A61B 5/374; A61B 5/383; A61B 5/384

(52) U.S. Cl. CPC A61B 5/4088; A61B 5/7267; A61B 5/7203; A61B 5/383; A61B 5/384; A61B 5/374

(57) ABSTRACT

An explainable artificial intelligence system includes a processor that visualizes and provides a diagnosed result, a decision-making structure which is description information for describing a basis for the diagnosis, a description of at least one second brain wave feature, and an importance of the at least one second brain wave feature.

### CROSS-REFERENCE TO RELATED APPLICATIONS

The present application is based upon and claims the benefit of priority to Korean Patent Application Nos. 10-2020-0155762, filed on Nov. 19, 2020, 10-2020-0156656, filed on Nov. 20, 2020, 10-2021-0036451 filed on Mar. 22, 2021, and 10-2021-0036452, filed on Mar. 22, 2021. The disclosures of the above-listed applications are hereby incorporated by reference herein in their entirety.

### TECHNICAL FIELD

Embodiments of the inventive concept described herein relate to an explainable artificial intelligence system for diagnosis of mental diseases, and more particularly, relate to an artificial intelligence system capable of being used in an electroceutical prescription for treating mental diseases a patient is suffering from using a machine learning model.

### BACKGROUND

People living in modern society are exposed to various situations, and there is a lot of mental stress caused due to this. Such stress causes mental diseases such as depression, and the resulting social problems are reaching a serious point.

Mental diseases are not limited to a specific age group and are developed, and the cause of the onset of the mental diseases is also diverse. Particularly, with the onset of COVID-19, the number of patients suffering from mental diseases such as anxiety disorders or depression is increasing rapidly as opportunities to communicate with each other between peoples decrease.

In the past, mental diseases of patients were identified through a self-response questionnaire or, according to the DSM-5 standard, a psychiatrist diagnosed the patient and identified the mental diseases he was suffering from, and then, performs drug treatment at the same time, if necessary. For example, the psychiatrist identifies mental diseases the patient is suffering from, using a self-response questionnaire such as The Beck Anxiety Inventory (BAI) or The State-Trait Anxiety Inventory (STAI) for anxiety disorders and identifies mental diseases using a self-response questionnaire such as The Beck Depression Inventory (BDI) for depression.

However, because evaluation criteria vary for each person varies although there is the same symptom in the self-response questionnaire, a different diagnosed result may be obtained. It is easy for the respondent to respond to increase or decrease his or her scores on purpose, there is a high possibility that diagnosed results different from actual results will be obtained according to the intention of the respondent. Furthermore, even when consulting with a doctor, people still have a reluctance to visit a psychiatrist according to the national sentiment, and it is difficult to visit a hospital due to their livelihood. In addition, when medication is used to treat mental diseases, it takes a considerable amount of time (usually 1 to 2 years) to be completely cured. There is a high possibility of causing many side effects such as nausea, vomiting, diarrhea, excitement, agitation, sleep disorder, the problem of sexual function, and headache.

Furthermore, when a medical person such as a psychiatrist makes a diagnosis, there is a possibility that an erroneous diagnosis result may be obtained due to his or her incomplete judgment.

The introduction of an artificial intelligence system to prevent incomplete judgment by medical person and to provide assistance to the medical person in diagnosis has been actively discussed. However, most artificial intelligence systems are in a black box structure which only provides decision-making results and is unable to explain the process or rationale leading to decision-making. No matter how high the artificial intelligence system having a structure incapable of explaining the process leading to decision-making, it is difficult for the artificial intelligence system to be actively used in the medical field where even the slightest mistake may have catastrophic consequences.

Thus, there has been a growing trend towards demands for electroceuticals for accurately identifying mental diseases the patient is suffering from, by means of the artificial intelligence system, and stimulating and treating a specific region of the brain.

### DISCLOSURE

### Technical Task

Embodiments of the inventive concept provide an explainable artificial intelligence system for diagnosing explainable mental diseases of a decision-making process leading to diagnosis of mental diseases.

Embodiments of the inventive concept provide an artificial intelligence system capable of being used to identify mental diseases a patient is suffering from by means of the analysis of the brain wave signal using a machine learning model and prescribe electroceuticals for treating the mental diseases.

### Technical Solution

According to an embodiment, an explainable artificial intelligence system may include a communication unit that receives a brain wave of a patient and a processor that preprocesses the received brain wave by means of noise cancellation and epoching processing, extracts at least one first brain wave feature from the preprocessed brain wave, determines at least one second brain wave feature necessary to diagnose mental diseases of the patient among the at least one first brain wave feature and a weight of at least one second brain wave feature, using a machine learning model learned for diagnosis of mental diseases, generates a decision-making structure for diagnosing the mental diseases of the patient, wherein an importance of the at least one second brain wave feature is determined in the process of generating the decision-making structure, substitutes the at least one second brain wave feature and the weight into the decision-making structure to diagnose the mental diseases of the patient, and visualizes and provides the diagnosed result, the decision-making structure which is description information for describing a basis for the diagnosis, a description of the at least one second brain wave feature, and the importance of the at least one second brain wave feature. The machine learning model may use a brain wave for each age and a brain wave feature for each channel, the brain wave feature being included in the brain wave for each age, as learning data to diagnose the mental diseases.

The description of the at least one second brain wave feature may include a channel name necessary to diagnose the mental diseases of the patient, a brain wave type and brain wave power in the channel, and connectivity between channels. The diagnosed result may be information about at least one mental disease the patient is suffering from.

The processor may compare any one of the at least one second brain wave feature with a threshold, in each stage of the decision-making structure and may determine a next comparison stage of comparing the second brain wave feature with the threshold, as a result of the comparison.

The processor may diagnose the mental diseases of the patient based on the weight and the at least one second brain wave feature in the lowest stage of the decision-making structure.

The machine learning model may extract the at least one second brain wave feature using an advanced variational autoencoder.

The machine learning model may be composed of an artificial neural network being in the form of an advanced variational autoencoder and is learned such that the at least one first brain wave feature is an input of the artificial neural network and such that the diagnosed result is output as an end result. The artificial neural network may be composed of a first recurrent neural network acting as an encoder and a second recurrent neural network acting as a decoder. The input of the first recurrent neural network may be the at least one first brain wave feature, the output of the first recurrent neural network may be the at least one second brain wave feature, the input of the second recurrent neural network may be the at least one second brain wave feature, and the output of the second recurrent neural network may be the diagnosed result.

A connection between the at least one first brain wave feature and a unit of the first recurrent neural network and a connection between the at least one second brain wave feature and a unit of the second recurrent neural network may be all-to-all linear connections. A connection weight may be randomly determined as uniform distribution. A value of the connection weight may be fixed in an initialization process and may then be not changed.

A connection between the unit of the first recurrent neural network and the at least one second brain wave feature and a connection between the unit of the second recurrent neural network and the diagnosed result may be the all-to-all linear connections. Values of connection weights of the connection between the unit of the first recurrent neural network and the at least one second brain wave feature and the connection between the unit of the second recurrent neural network and the diagnosed result may be changed while learned by a linear learning algorithm.

The values of the connection weights of the connection between the unit of the first recurrent neural network and the at least one second brain wave feature and the connection between the unit of the second recurrent neural network and the diagnosed result may be randomly determined as uniform distribution and may then be changed while learned by the linear learning algorithm.

The generating of the decision-making structure may include representing a result learned from the first recurrent neural network to the at least one second brain wave feature, the second recurrent neural network, and the diagnosed result as a first formula in the machine learning model and converting the represented first formula into the decision-making structure.

The importance of the at least one second brain wave feature may be determined in the process of converting the decision-making structure. The importance of the at least one second brain wave feature may be obtained by digitizing an influence of the second brain wave feature to diagnose the mental diseases of the patient.

The first recurrent neural network may be composed of a plurality of units. The number of the units making up the first recurrent neural network may be determined to be greater than 100 times the number of the at least one first brain wave feature.

The units making up the first recurrent neural network are randomly and recurrently connected with each other and a connection probability between the respective units may be determined between from 0.1% to 1%. A connection weight between the units making up the first recurrent neural network may be determined as uniform distribution among values between from −1 to 1 and a certain scaling factor may be multiplied by connection weights such that an absolute value of the largest eigen value of a connection weight matrix determined subsequently becomes 1 or less. The calculated connection weight matrix may be subsequently fixed and may not be changed.

The learning data may further include feedback information of a medical team about the diagnosis of the mental diseases.

The processor may determine at least one brain region corresponding to the diagnosed result among cerebral regions of the patient, may generate stimulation information for stimulating at least one stimulation channel to stimulate a cerebral cortex of the at least one determined brain region and provides the generated stimulation information, and may further visualize and provide the at least one stimulation channel other than the diagnosed result, the decision-making structure, the description of the at least one second brain wave feature, and the importance of the at least one second brain wave feature. The machine learning model may further use a treatment progress according to stimulation for each region of the cerebrum as learning data other than the brain wave for each age and the brain wave feature for each channel, the brain wave feature being included in the brain wave for each age, to diagnose the mental diseases.

According to an embodiment, a control method of an explainable artificial intelligence system may include receiving a brain wave of a patient, preprocessing the received brain wave by means of noise cancellation and epoching processing, extracting at least one first brain wave feature from the preprocessed brain wave, determining at least one second brain wave feature necessary to diagnose mental diseases of the patient among the at least one first brain wave feature and a weight of the at least one second brain wave feature, using a machine learning model learned for diagnosis of mental diseases, generating a decision-making structure for diagnosing the mental diseases of the patient, wherein an importance of the at least one second brain wave feature is determined in the process of generating the decision-making structure, substituting the at least one second brain wave feature and the weight into the decision-making structure to diagnose the mental diseases of the patient, and visualizing and providing the diagnosed result, the decision-making structure which is description information for describing a basis for the diagnosis, a description of the at least one second brain wave feature, and the importance of the at least one second brain wave feature. The machine learning model may use a brain wave for each age and a brain wave feature for each channel, the brain wave feature being included in the brain wave for each age, as learning data to diagnose the mental diseases.

According to an embodiment, a computer-readable storage medium may store a program for implementing the control method of the explainable artificial intelligence system.

### Technical Effect

According to embodiments disclosed in the inventive concept, the explainable artificial intelligence system may be used in the medical field as it is possible to explain a decision-making process leading to diagnosis of mental diseases to the user.

According to embodiments disclosed in the inventive concept, the explainable artificial intelligence system may identify mental diseases the patient is suffering from by means of the analysis of brain wave signals and may prescribe electroceuticals for treating the mental diseases.

### BRIEF DESCRIPTION OF THE FIGURES

FIG. 1 is a schematic diagram illustrating collecting learning data according to an embodiment of the inventive concept;

FIG. 2 is a block diagram illustrating an XAI system according to an embodiment of the inventive concept;

FIG. 3 is a schematic diagram illustrating diagnosing mental diseases using an XAI system according to an embodiment of the inventive concept;

FIGS. 4A, 4B, 4C, 4D, 4E, and 4F are schematic diagrams illustrating diagnosing mental diseases and prescribing electroceuticals using an XAI system according to an embodiment of the inventive concept;

FIG. 5 is a drawing illustrating a decision-making structure provided by an XAI system according to an embodiment of the inventive concept;

FIG. 6 is a drawing illustrating a stimulation channel provided by an XAI system according to an embodiment of the inventive concept; and

FIG. 7 is a flowchart illustrating diagnosing mental diseases and prescribing electroceuticals using an XAI system according to an embodiment of the inventive concept.

### DETAILED DESCRIPTION

Advantages, features, and methods of accomplishing the same will become apparent with reference to embodiments described in detail below together with the accompanying drawings. However, the inventive concept is not limited by embodiments disclosed hereinafter, and may be implemented in various forms. Rather, these embodiments are provided to so that this disclosure will be through and complete and will fully convey the concept of the invention to those skilled in the art, and the inventive concept will only be defined by the appended claims.

The terms used herein are provided to describe embodiments, not intended to limit the inventive concept. In the specification, the singular forms include plural forms unless particularly mentioned. The expressions “comprise” and/or “comprising” used herein indicate existence of one or more other elements other than stated elements but do not exclude presence of additional elements. Like reference numerals designate like elements throughout the specification, and the term “and/or” may include each of stated elements and one or more combinations of the stated elements. The terms such as “first” and “second” are used to describe various elements, but it is obvious that such elements are not restricted to the above terms. These terms are only used to distinguish one component from another component. Thus, it is obvious that a first element described hereinafter may be a second element within the technical scope of the inventive concept.

The word “exemplary” is to mean serving as an example, instance, or illustration in the specification. Any embodiment described in the specification as “exemplary” is not necessarily to be construed as preferred or advantageous over other embodiments.

Furthermore, the term “unit” as used herein means, but is not limited to, a software or hardware component, such as field-programmable gate array (FPGA) or application-specific integrated circuit (ASIC), which performs certain tasks. However, the “unit” is not limited to software or hardware. The “unit” may be configured to reside on the addressable storage medium and configured to execute on one or more processors. Thus, as an example, the “unit” may include elements, such as software elements, object-oriented software elements, class elements and task elements, processes, functions, attributes, procedures, subroutines, segments of program codes, drivers, firmware, microcode, circuitry, data, databases, data structures, tables, arrays, and variables. The functionality provided for in the elements and “unit” or may be combined into fewer elements and “units” or further separated into additional elements and “units”.

Furthermore, all “units” of the specification may be controlled by at least one processor, and at least one processor may perform an operation performed by the “unit” of the inventive concept.

Embodiments of the inventive concept may be described in terms of a function or a block performing the function. The block, which may be referred to herein as the ‘unit’ or ‘module’ of the inventive concept is physically implemented by analog and/or digital circuits such as logic gates, integrated circuits, microprocessors, microcontrollers, memories, passive electronic components, active electronic components, optical components, and hardwired circuits, and may optionally be driven by firmware and software.

An embodiment of the inventive concept may be implemented using at least one software program run on at least one hardware device and may perform a network management function for controlling elements.

Unless otherwise defined herein, all terms (including technical and scientific terms) used in the specification may have the same meaning that is generally understood by a person skilled in the art. Also, terms which are defined in a dictionary and commonly used should be interpreted as not in an idealized or overly formal detect unless expressly so defined.

Hereinafter, an embodiment of the inventive concept will be described in detail with reference to the accompanying drawings.

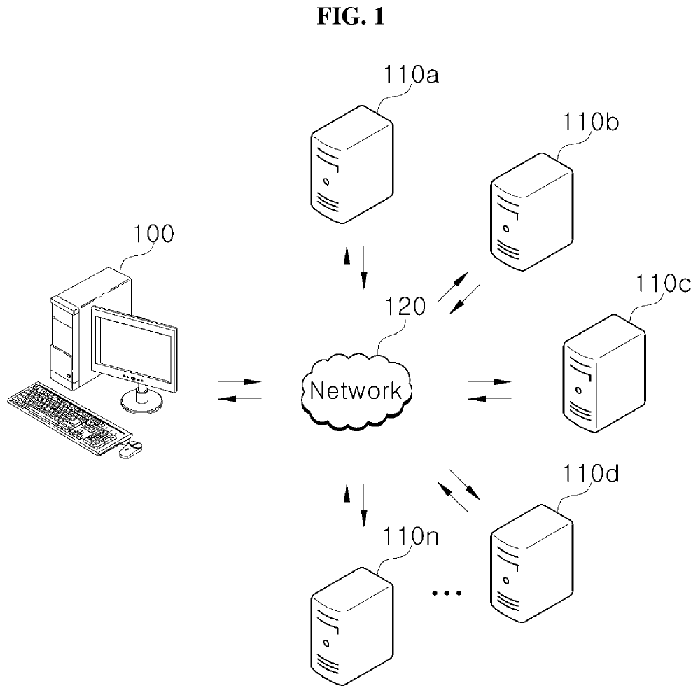

FIG. 1 is a schematic diagram illustrating collecting learning data according to an embodiment of the inventive concept.

An explainable artificial intelligence (XAI) system 100 according to the inventive concept may diagnose mental diseases of a patient, may provide the diagnosed result and a diagnostic basis together, and may additionally provide a stimulation channel for treating mental diseases. The XAI system 100 may communicate with at least one server 110a to 110n to collect learning data for learning. In this case, the at least one server 110a to 110n may include a cloud server such as Amazon Web Services (AWS) or MS Azure. Furthermore, the at least one server 110a to 110n may include a calculation service which calculates a brain wave signal to analyze the brain wave signal and identify mental diseases the patient is suffering from. The XAI system 100 may obtain analysis data of various brain wave signals, mental diseases corresponding to a specific brain wave signal, and the like from the at least one server 110a to 110n.

The XAI system 100 may communicate with the at least one server 110a to 110n using a network 120. The network 120 may include a connection unit (not shown) such as a wired communication link, a wireless communication link, or an optical fiber cable. Furthermore, the network 120 may be implemented with several various networks such as an intranet and a local area network (LAN) or a wide area network (WAN).

The XAI system 100 according to the inventive concept may diagnose mental diseases of the patient by means of a decision-making structure and a brain wave using a machine learning model, such as deep learning, which is learned for diagnosis of mental diseases. Furthermore, the XAI system 100 may determine a brain region corresponding to the diagnosed mental diseases among cerebral regions of the patient and may generate and provide stimulation information for stimulating a channel to stimulate the cerebral cortex of the determined brain region.

The deep learning may refer to a machine learning method based on an artificial neural network, which allows a machine to simulate and human biological neurons.

As an example of the machine learning model, a deep neural network (DNN) may include a system or a network which constructs one or more layers in one or more computers and performs determination based on a plurality of data.

The DNN may be implemented with a set of layers including a convolutional pooling layer, a locally-connected layer, and a fully-connected layer.

The convolutional pooling layer or the locally-connected layer may be configured to extract features of the brain wave. The fully-connected layer may determine a correlation between brain wave features.

For another example, the entire structure of the DNN according to the inventive concept may be implemented in a form where the locally-connected layer is connected with the convolutional pooling layer and the fully-connected layer is connected with the locally-connected layer. The DNN may include various determination criteria (i.e., parameters) and may add a new determination criterion (i.e., a parameter) by analyzing the input brain wave.

The DNN according to embodiments of the inventive concept may be a structure called a convolutional neural network, which may be configured in a structure in which a feature extraction layer for learning a feature with the largest discriminative power by itself from given image data and a prediction layer for learning a prediction model to have the highest prediction performance based on the extracted feature are integrated with each other.

The feature extraction layer may be formed in a structure where a convolution layer for generating a feature map by applying a plurality of filters and a pooling layer capable of extracting a feature which is invariant to a change in position or rotation are alternately repeated several times. As a result, various levels of features from a low level of features such as a point, a line, or a surface to a high level of features which are complicated and meaningful may be extracted.

The convolution layer obtains a feature map by taking a non-linear activation function from the inner product of the filter and the local receptive field with respect to each patch of the input. Compared with another network structure, the CNN has a feature using a filter having sparse connectivity and shared weights. Such a connection structure reduces the number of parameters to learn and makes learning through the backpropagation algorithm efficient, and the prediction performance is consequently improved.

As a classification model such as multi-layer perception (MLP) or support vector machine (SVM) is combined in the form of a fully-connected layer, the feature finally extracted through repetition of the convolution layer and the pooling layer may be used to learn and predict the classification model.

Furthermore, according to an embodiment of the inventive concept, learning data for machine learning may be generated based on a U-Net-dhSgement model. Herein, the U-Net-dhSgement model may be a model which sets an expansive path to be symmetrical to a contracting path and generates a U-shaped architecture having a skip connection for each level, based on end-to-end fully convolutional networks (FCN).

According to an embodiment of the inventive concept, the machine learning model may be learned to diagnose mental diseases using learning data including at least one of a brain wave for each age, analysis data of the brain wave, and mental diseases corresponding to a feature of the brain wave. The machine learning model may use a brain wave for each age, a preprocessed brain wave feature included in the brain wave for each age, and mental diseases corresponding to a brain wave feature for each channel among the brain wave features as learning data. In detail, the machine learning model may use mental diseases of a patient, which is finally derived as the diagnosed result by substituting the brain waver feature for each channel into a decision-making structure which will be described below, as learning data.

Furthermore, the machine learning model according to the inventive concept may use a cerebral region corresponding mental diseases, treatment progress according to stimulation for each region of the cerebrum, or the like as learning data. As a result, the XAI system 100 may learn a treatment effect of a brain region corresponding to a brain wave signal and mental diseases using the learning data for the purpose of treating mental diseases of another patient.

Furthermore, the machine learning model may use feedback information of a medical team about diagnosis of mental diseases as learning data. When the XAI system 100 provides the diagnosed result, the decision-making structure which is the diagnosis basis, and the stimulation channel as visual information, the medical team may determine whether to trust the information depending on his or her own judgment. When it is determined that the diagnosed result or the decision-making structure of the XAI system 100 is wrong, the medical team may correct the wrong portion and may provide the XAI system 100 with the corrected information as feedback information. The XAI system 100 may use the feedback information of the medical team as learning data. Furthermore, when the diagnosed result or the decision-making structure of the XAI system 100 is valid, but when the XAI system 100 wrongly determines a brain region to stimulate and wrongly provides a stimulation channel, a stimulation intensity, a stimulation period, or the like, the medical team may correct a wrong portion and may provide the XAI system 100 with the corrected information as feedback information. The XAI system 100 may use the feedback information of the medical team as learning data.

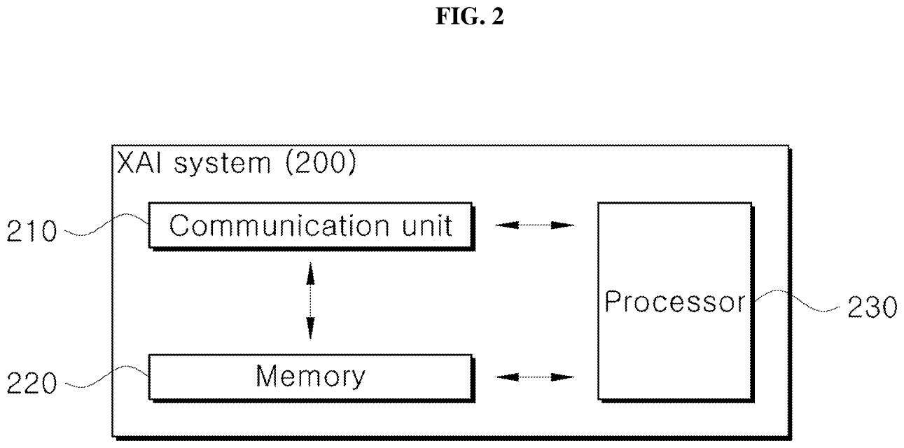

FIG. 2 is a block diagram illustrating an XAI system 200 according to an embodiment of the inventive concept. The XAI system 200 of FIG. 2 may correspond to an XAI system 100 of FIG. 1.

According to an embodiment, the XAI system 200 may include a communication unit 210, a memory 220, and a processor 230. As components shown in FIG. 2 are not essential in implementing the XAI system 200, the XAI system 200 described on the specification may have components greater or less than the components listed above. For example, the communication unit 210 among the components may include one or more modules capable of performing wireless communication with an external device (not shown) or an external server.

A brain wave refers to the recording of potentials on the vertical axis and time on the horizontal axis by attaching electrodes to the scalp to induce minute electrical activity of brain cell populations and amplifying it using an electroencephalograph. In other words, brain wave measurement is measuring electrical activity generated in the cerebral cortex. The brain wave may change in time and space depending on activity of the brain, a state upon measurement, and a brain function and may mainly have a frequency of 0 Hz to 50 Hz and an amplitude of 10 uV to 200 uV. Furthermore, the brain wave may be classified as a delta (δ) wave, a theta (θ) wave, an alpha (α) wave, a beta (β) wave, or the like. A feature of each brain wave may be present for each frequency.

According to an embodiment of the inventive concept, EEG refers to electroencephalogram and refers to an electrical recording signal recorded by inducing potential fluctuations occurring in the brain of a person or animal or a brain current occurring by it on the scalp. MEG refers to magnetoencephalogram and refers to a signal recorded by measuring minute biomagnetism generated by the electrical activity of nerve cells in the brain using a SQUID sensor or the like. ECoG refers to electrocorticogram and refers to an electrical recording signal recorded by implanting electrodes from the surface of the cerebral cortex and directly measuring potential fluctuations occurring in the cerebrum or a brain current caused by it. NIRS refers to near-infrared spectroscopy. An NIRS brain wave signal used in the inventive concept refers to a signal recorded by measuring a difference between low-level light waves being reflected from the brain. Brain wave signals such as EEG, MEG, and ECoG are exemplified in the specification. However, the brain wave signal is not limited to the specific type of brain wave signal, which may refer to all signals capable of being measured from the human head.

According to an embodiment of the inventive concept, the brain wave of a patient may be measured using portable brain wave measurement equipment such as Emotiv, OpenBci, or NeuroSky. In this case, the patient may measure his or her brain wave using his or her own portable brain wave measurement equipment to transmit the brain wave to a hospital or may be provided with brain wave measurement equipment from the hospital and may measure his or her own brain wave to transmit the brain wave to the hospital. As a result, the patient may measure his or her own brain wave to be diagnosed with mental diseases in a time or place he or she wants without going to the hospital In a non-face-to-face manner. The measured brain wave may be received through the communication unit 210 of the XAI system 200.

Furthermore, when the patient visits the hospital in person, the brain wave signal of the patient may be measured by a head cap manufactured by Biosemi (registered trademark) on which 64 electrodes are mounted. For example, the medical team in the hospital may measure a brain wave of the patient, using a Geodesic™ brain wave measurement device (not shown) sold by Electrical Geodesics Inc. (EGI) (registered trademark) or another type of brain wave measurement device (not shown) such as those sold by Compumedics NeuroScan (registered trademark), which is usually performs calculation among 16 and 256 electrodes. When the patient visits the hospital in person, the XAI system 200 may receive the brain wave of the patient, which is measured in the hospital, through the communication unit 210.

The communication unit 210 according to the inventive concept may communicate with various types of external devices depending on various types of communication schemes. The communication unit 210 may include at least one of a wireless-fidelity (Wi-Fi) chip, a Bluetooth chip, a wireless communication chip, or a near field communication (NFC) chip.

According to a mobile communication technology of the specification, the communication unit 210 may transmit and receive a wireless signal with at least one of a base station, an external terminal, or an external server on a mobile communication network established according to technical standards or a communication scheme (e.g., global system for mobile communication (GSM), code division multi access (CDMA), code division multi access 2000 (CDMA2000), enhanced voice-data optimized or enhanced voice-data only (EV-DO), wideband CDMA (WCDMA), high speed downlink packet access (HSDPA), high speed uplink packet access (HSUPA), long term evolution (LTE), long term evolution-advanced (LTE-A), or the like).

Furthermore, the wireless Internet technology of the specification may be, for example, wireless LAN (WLAN), wireless-fidelity (Wi-Fi), Wi-Fi Direct, digital living network alliance (DLNA), wireless broadband (WiBro), world interoperability for microwave access (WiMAX), high speed downlink packet access (HSDPA), high speed uplink packet access (HSUPA), long term evolution (LTE), long term evolution-advanced (LTE-A), or the like.

Furthermore, the short range communication technology of the specification may include a technology supporting short range communication using at least one of Bluetooth™, radio frequency identification (RFID), infrared data association (IrDA), ultra wideband (UWB), ZigBee, near field communication (NFC), wireless-fidelity (Wi-Fi), Wi-Fi Direct, and wireless universal serial bus (USB) technologies.

According to an embodiment of the inventive concept, the memory 220 is a local storage medium for supporting various functions of the XAI system 200. The memory 220 may include a brain wave signal received by the communication unit 210 and may store analysis data of the brain wave signal, a type of mental diseases corresponding to a specific brain wave signal, or the like. Furthermore, the memory 220 may store a plurality of application programs or applications run in the XAI system 200, data for an operation of the XAI system 200, and instructions. At least some of such application programs may be downloaded from the external server through wireless communication. The application program may be stored in the memory 220, may be installed on the XAI system 200, and may be run to perform an operation (or function) of the XAI system 200 by the processor 230.

Furthermore, the memory 220 according to the inventive concept may be provided as a writable ROM, such that pieces of data should remain although power supplied to the XAI system 200 is cut off, to reflect changes. In other words, the memory 220 may be provided as one of a flash memory, an erasable programmable ROM (EPROM), or an electrically erasable programmable ROM (EEPROM). The inventive concept describes that all pieces of instruction information are stored in the one memory 220 for convenience of description, but not limited thereto. The XAI system 200 may have a plurality of memories.

The processor 230 may generally control the overall operation of the XAI system 200 other than an operation associated with the application program. The processor 230 may process a signal, data, information, or the like input or output through the components described above or may run the application program stored in the memory 220, thus providing or processing information or a function suitable for a user.

Furthermore, the processor 230 may control at least some of the components of FIG. 2 to run the application program stored in the memory 220. In addition, the processor 230 may combine and operate at least two or more of the components included in the XAI system 200 to run the application program.

Hereinafter, the operation of the processor 230 of the XAI system 200 will be described with reference to FIGS. 2 to 6.

According to an embodiment of the inventive concept, when the brain wave of the patient is received through the communication unit 210, the processor 230 may perform preprocessing.

In general, an interval where noise occurs may be generated due to a heartbeat or movement of the body when the brain wave is measured. Thus, the processor 230 may perform a preprocessing process of removing a high-frequency component and a low-frequency component, which are unnecessary to diagnose mental diseases by means of brain wave analysis, and removing an artifact due to motion.

According to an embodiment of the inventive concept, the processor 230 may preprocess the brain wave received by means of noise cancellation or filtering. In detail, the processor 230 may use independent component analysis, principle component analysis, or the like for removing electromyography (EMG) or electrooculogram (EOG) noise to remove noise.

Furthermore, the processor 230 may remove noise using any one of a low-pass filter, a high-pass filter, a band-pass filter, or a notch filter. For example, because other biometric signals except for a brain wave signal such as electromyogram (EMG) or electrooculogram (EOG) are signals of interest, other than a normal noise signal according to a general transmission path (a wired or wireless channel), the processor 230 may treat them as noise to remove them by means of filtering or the like.

Furthermore, according to an embodiment of the inventive concept, epoching processing refers to cutting brain wave data, noise of which is removed, into a specific interval to perform signaling. Epoching may be used in tens of milliseconds to seconds.

According to an embodiment of the inventive concept, the processor 230 may extract at least one brain wave feature from the preprocessed brain wave. In this case, the processor 230 may extract power for each frequency by means of spectral density analysis and may extract a quantitative brain wave feature using a linear or non-linear network analysis, complex system network analysis, or the like.

In detail, the processor 230 may extract a brain wave feature using a technology such as Fourier transform, partial directed coherence (PDC), direct transfer function (DTF), independent component analysis (ICA), principle component analysis (PCA), or common spatial pattern (CSP). Furthermore, the brain wave feature may be obtained from event related potential (ERP), steady state visually evoked potential (SSVEP), event related synchronization (ERS), event related desynchronization (ERD), default mode network (DMN), or a combination thereof.

According to an embodiment of the inventive concept, a first brain wave extracted by means of the processor 230 may include brain wave (e.g., γ wave, α wave, β wave, δ wave, θ wave, or the like) power for each frequency of a patient, a frequency, connectivity between channels, and the like. Herein, the channel may include a plurality of points of the scalp where the brain wave of the patient is measured. Furthermore, the connectivity between channels may include a phase locking value (PLV) between brain wave signals, a correlation coefficient, a coherence coefficient, a Granger's Causality Index, partial directed coherence (PDC), a directed transfer function (DTF), mutual Information, transfer entropy, synchronization likelihood, and the like.

When at least one first brain wave feature is extracted, the processor 230 may diagnose mental diseases by means of at least one second brain wave feature necessary to diagnose mental diseases of a patient and a weight of at least one second brain wave feature, using a machine learning model learned for diagnosis of mental diseases. Furthermore, the processor 230 may determine a brain region corresponding to the diagnosed mental diseases among cerebral regions of the patient as a treatment region.

Furthermore, according to an embodiment of the inventive concept, the processor 230 may determine the decision-making structure as description information for describing the diagnosed result and a basis for diagnosis and may visualize the generated decision-making structure to provide visual information to the medical team.

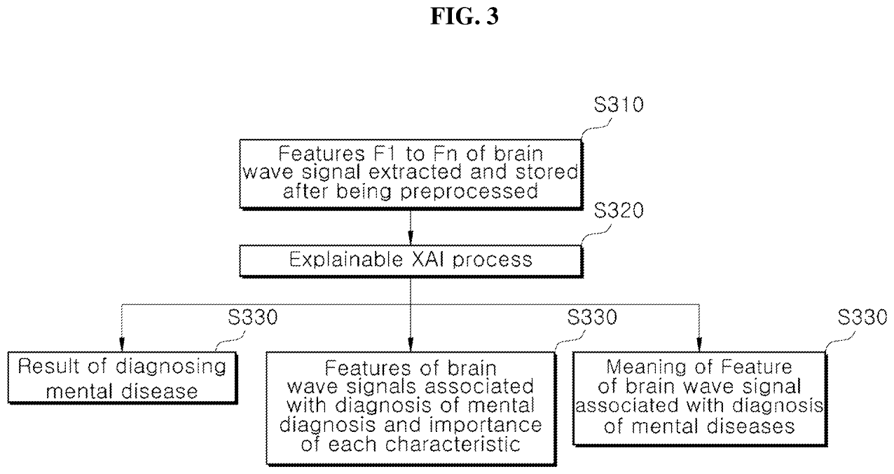

FIG. 3 is a schematic diagram illustrating diagnosing mental diseases using an XAI system according to an embodiment of the inventive concept.

First of all, in S310, a first brain wave of F1 to Fn may be extracted through a preprocessing and feature extraction process of a brain wave of a patient.

Next, in S320, an XAI system 200 may receive the first brain wave signal and may diagnose mental diseases of the patient through an explainable XAI process. Herein, the explainable XAI process may refer to a series of processing of diagnosing mental diseases using the machine learning model of the specification.

When the diagnosis of the mental diseases of the patient is completed, in S330, the XAI system 200 may visualize the diagnosed result, a brain wave signal associated with diagnosis of mental diseases, and importance information about the brain wave signal associated with the diagnosis to provide a medical team with the visualized information.

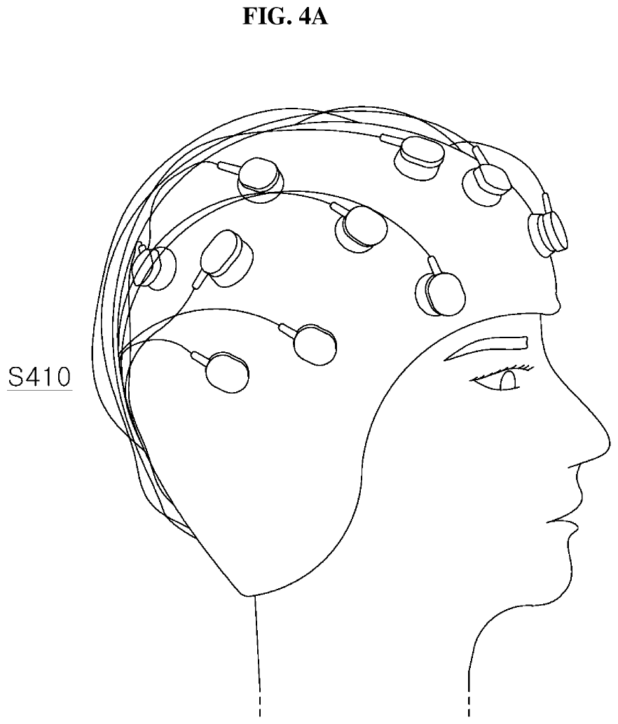

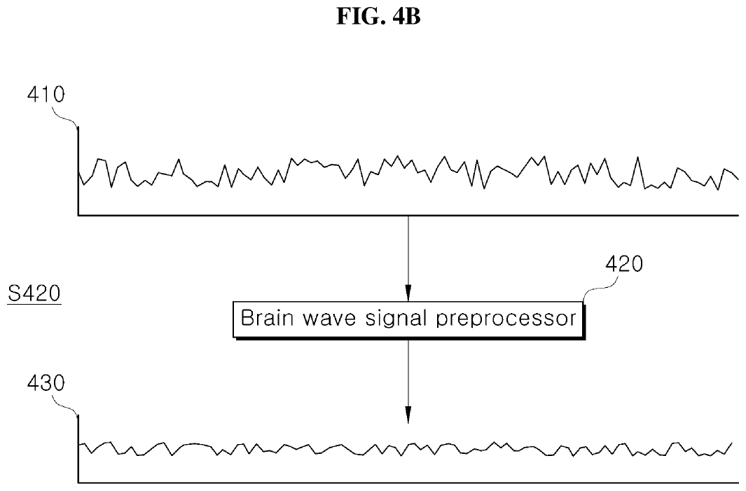

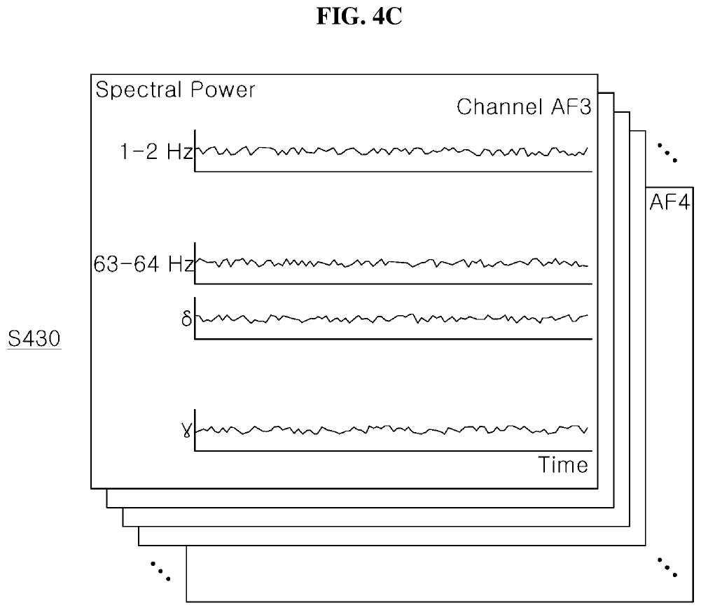

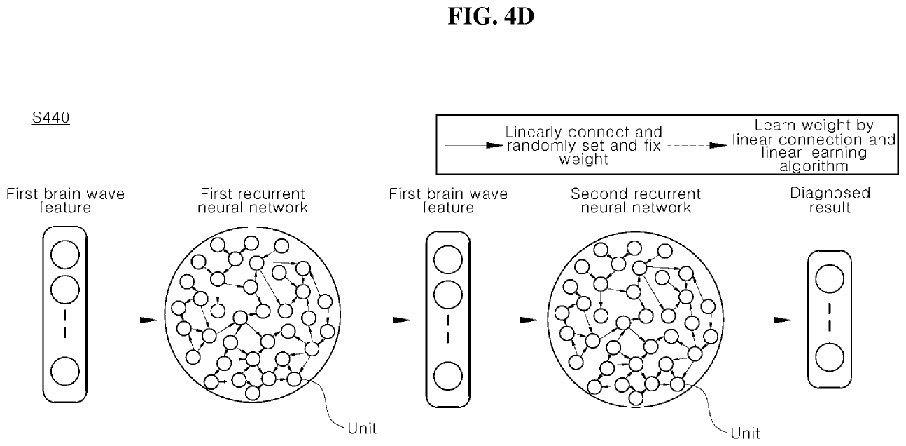

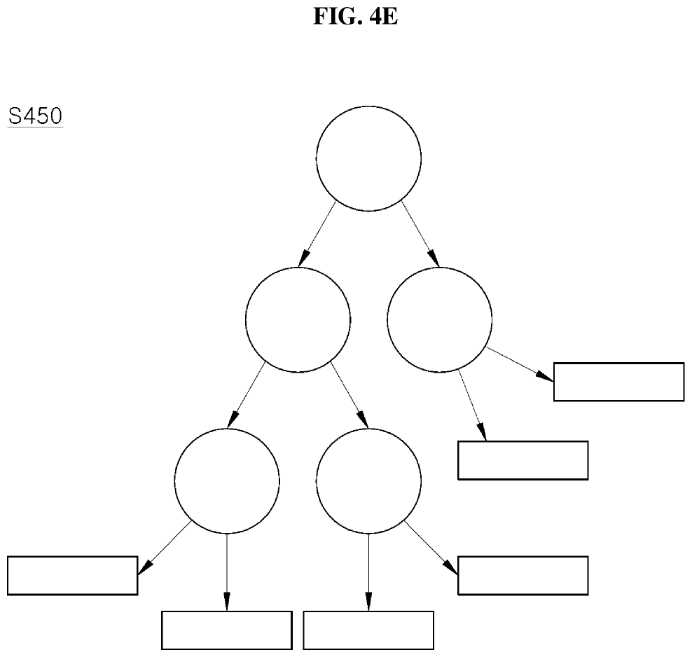

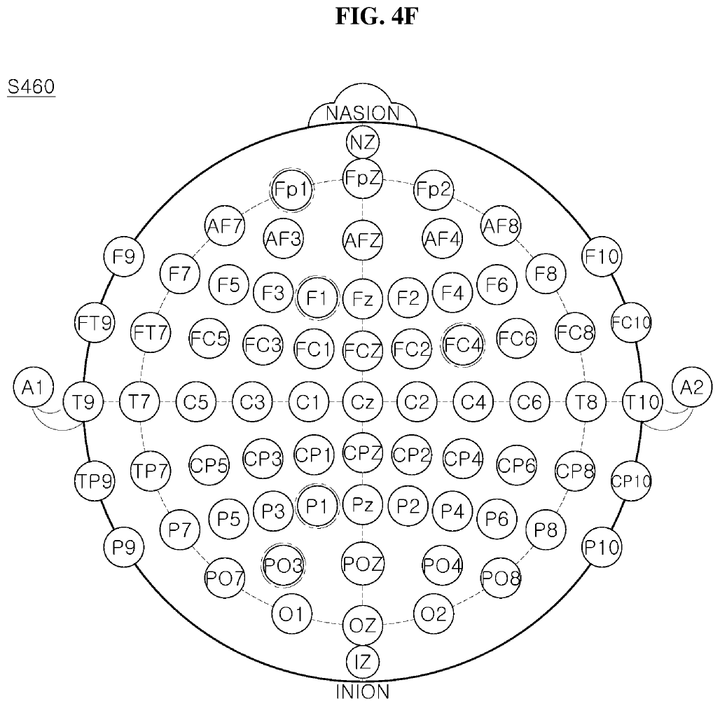

Hereinafter, the process of diagnosing mental diseases will be described in detail with reference to FIGS. 4A to 4F.

FIGS. 4A to 4F are schematic diagrams illustrating diagnosing mental diseases using an XAI system according to an embodiment of the inventive concept.

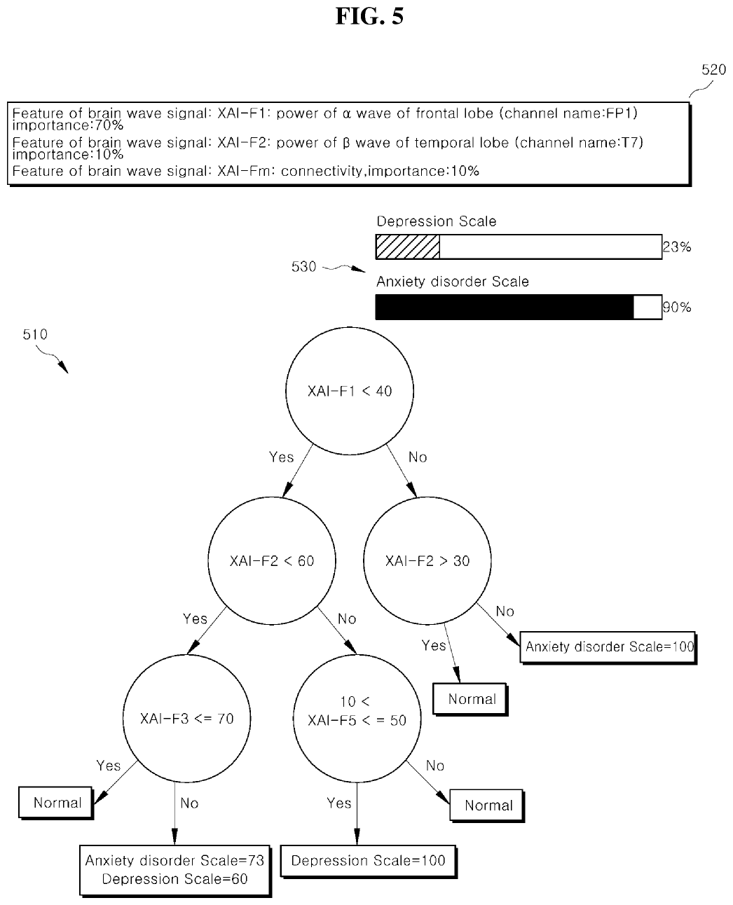

FIG. 5 is a drawing illustrating a decision-making structure provided by an XAI system according to an embodiment of the inventive concept.

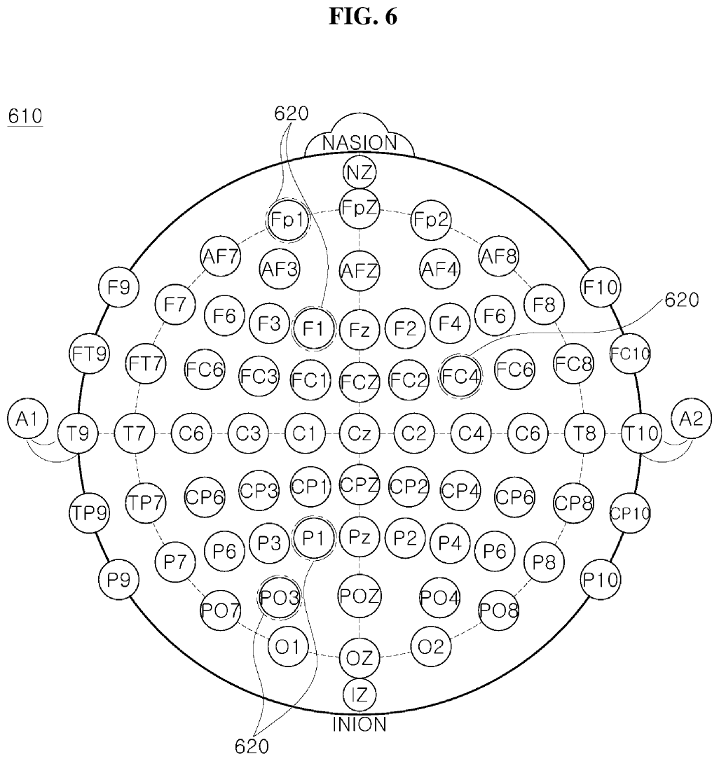

FIG. 6 is a drawing illustrating a stimulation channel provided by an XAI system according to an embodiment of the inventive concept.

First of all, referring to FIG. 4A, in S410, a brain wave of a patient may be measured by means of brain wave measurement equipment.

The brain wave of the patient may be measured by the brain wave measurement equipment such as Emotiv, OpenBci, NeuroSky, or Geodesic™ and may be received in a communication unit 210 of an XAI system 200 of FIG. 2 over a network.

Referring to FIG. 4B, in S420, the received brain wave 410 may be preprocessed by means of noise cancellation and epoching processing.

In detail, the received brain wave 410 may be preprocessed by means of noise cancellation and epoching processing by a brain wave signal preprocessor 420 which removes noise using at least one of a low-pass filter, a high-pass filter, a band-pass filter, and a notch filter. In this case, when the XAI system 200 includes the brain wave signal preprocessor 420, a processor 230 of FIG. 2 may control the brain wave signal preprocessor 420 to perform preprocessing. Alternatively, when the brain wave signal preprocessor 420 is an external device outside the XAI system 200, the XAI system 200 may receive a brain wave 430 preprocessed by the brain wave signal preprocessor 420 through the communication unit 210.

Referring to FIG. 4C, when the preprocessing is completed, in S430, the processor 230 may extract at least one first brain wave feature from the preprocessed brain wave 430.

In detail, the processor 230 may extract power for each frequency by means of spectral density analysis and may extract a quantitative brain wave feature using a linear or non-linear network analysis, complex system network analysis, or the like.

Next, referring to FIG. 4D, in S440, the processor 230 may input the at least one extracted first brain wave to a machine learning model learned for diagnosis of mental diseases to diagnose mental diseases.

In this case, at least one of an age of a patient, a mental state of the patient, which is identified by means of a self-response questionnaire, and health status information of the patient, which is identified by a medical team may be additionally input to the machine learning model.

According to an embodiment of the inventive concept, the processor 230 may determine at least one second brain wave feature necessary to diagnose mental diseases of the patient among the at least one first brain wave feature and a weight of at least one second brain wave feature.

Herein, the second brain wave may include brain wave (e.g., γ wave, α wave, β wave, δ wave, θ wave, or the like) power for each frequency of the patient, a frequency, connectivity between channels, and the like.

The processor 230 may use a machine learning model learned through an advanced variational autoencoder which will be described below to determine the at least one second brain wave feature necessary to diagnose mental diseases of the patient among the at least one first brain wave feature.

Herein, a description will be given of the machine learning model learned by means of the advanced variational autoencoder according to an embodiment of the inventive concept with reference to FIG. 4D.

The machine learning model according to an embodiment of the inventive concept may be composed of an artificial neural network which is in the form of an advanced variational autoencoder and may be learned such that the at least one first brain wave feature extracted from the preprocessed brain wave 430 is an input of the present artificial neural network and such that the diagnosed result is output as the end result.

In detail, the present artificial neural network may be composed of a first recurrent neural network acting as an encoder and a second recurrent neural network acting as a decoder. The input of the first recurrent neural network may be the at least one first brain wave feature. The output of the first recurrent neural network may be the at least one second brain wave feature. The input of the second recurrent neural network may be the at least one second brain wave feature. The output of the second recurrent neural network may be the diagnosed result.

The first recurrent neural network may be composed of a plurality of units. The number (N) of the units making up the first recurrent neural network may be determined by the number (M) of first brain wave features. For example, the number (N) of the units making up the first recurrent neural network may be determined by the processor 230 to be greater than 100 times the number (M) of the first brain wave features (N>M*100).

Herein, when the number (N) of the units making up the first recurrent neural network increases, because performance capable of being performed by the artificial neural network increases, but because the learning process takes a long time, the criterion of the number (N) of the units making up the first recurrent neural network in the present artificial neural network is set to a degree greater than 100 times the number (M) of the first brain wave feature.

Furthermore, the units making up the first recurrent neural network may be randomly and recurrently connected with each other. In this case, the processor 230 may determine a probability of being connected between the respective units, for example, between from 0.1% to 1%.

In addition, a connection weight between the units making up the first recurrent neural network may be determined as uniform distribution among, for example, values between from −1 to 1. The processor 230 may multiply connection weights by a certain scaling factor such that an absolute value of the largest eigen value of a connection weight matrix W subsequently determined becomes, for example, 1 or less.

The connection weight matrix W calculated in the above manner is fixed and is not changed.

A second recurrent neural network may be composed according to the same condition as the first recurrent neural network. The connection weight matrix W calculated for the second recurrent neural network is also fixed and is not changed.

Meanwhile, a connection between the first brain wave feature and the unit of the first recurrent neural network and a connection between the second brain wave feature and the unit of the second recurrent neural network may be all-to-all linear connections. The connection weight may be randomly determined as uniform distribution among, for example, values between from −1 to 1. The value of the connection weight is fixed in the initialization process and is then not changed.

In addition, a connection between the unit of the first recurrent neural network and the second brain wave feature and a connection between the unit of the second recurrent neural network and the diagnosed result may be all-to-all linear connections. A connection weight of the connection between the unit of the first recurrent neural network and the second brain wave feature and the connection between the second recurrent neural network and the diagnosed result in the initialization process may be randomly determined as uniform distribution among, for example, values between from −1 to 1.

However, a value of the connection weight of the connection between the unit of the first recurrent neural network and the second brain wave feature and the connection between the unit of the second recurrent neural network and the diagnosed result is not fixed to a value determined in the initialization process and is changed while being learned by a linear learning algorithm. For example, a linear regression or pseudo inverse matrix scheme may be used as the linear learning algorithm.

When the machine learning model is composed of an artificial neural network which is in the form of an autoencoder or a variational autoencoder rather than the advanced variational autoencoder, as complexity of calculation increases because the connection relationship is not a linear connection, a running rule is complicated. In addition, an artificial neural network which is in the form of a variational autoencoder or an autoencoder also follows machine constraints of having to using a machine with high performance depending on complexity of the running rule.

On the other hand, the machine learning model according to an embodiment of the inventive concept may randomly set and fix weights of some components based on the linear connection and may learn only weights of the second brain wave feature and the diagnosed result using the linear learning algorithm, thus reducing complexity of calculation and obtaining high accuracy based on a reservoir computing scheme.

In addition, because of using the linear connection and the linear learning algorithm, the XAI system 200 according to an embodiment of the inventive concept may reduce the amount of learning and may calculate an explainable result. In detail, because the results learned from the first recurrent neural network to the second brain wave feature, the second recurrent neural network, and the diagnosed result are linearly connected with each other in FIG. 4D, the XAI system 200 according to an embodiment of the inventive concept may represent the results learned from the first recurrent neural network to the second brain wave feature, the second recurrent neural network, and the diagnosed result as a first formula by means of a filter and may represent the derived first formula as a decision-making structure (a decision tree) which will be described below.

According to an embodiment of the inventive concept, the processor 230 may determine a weight for each of the second brain wave features together using the machine learning model, as well as the at least one second brain wave feature.

When the second brain wave feature and the weight are determined, in S450, the processor 230 may diagnose mental diseases of the patient by means of the decision-making structure.

In the inventive concept, the decision-making structure may be a series of processes where the processor 230 sequentially performs consideration, comparison, and calculation to diagnose mental diseases of the patient.

According to an embodiment of the inventive concept, the decision-making structure may be a tree structure where the mental diseases of the patient are diagnosed based on a weight and the at least one second brain wave feature in the lowest stage.

The processor 230 may generate a decision-making structure for diagnosing mental diseases of the patient and may substitute at least one second brain wave feature and a weight into the decision-making structure to diagnose the mental diseases of the patient.

In detail, as described above, because the results learned from the first recurrent neural network to the second brain wave feature, the second recurrent neural network, and the diagnosed result are linearly connected with each other in FIG. 4D, the XAI system 200 according to an embodiment of the inventive concept may represent the results learned from the first recurrent neural network to the second brain wave feature, the second recurrent neural network, and the diagnosed result as a first formula by means of a filter and may represent the derived first formula as a decision-making structure (a decision tree) which will be described below.

Meanwhile, the XAI system 200 according to an embodiment of the inventive concept may determine an importance of each second brain wave feature in the process of representing the learned result as a first linear formula and changing and representing the learned result as a decision-making structure.

The importance of the second brain wave feature may be provided together with the decision-making structure to understand the decision-making structure and is about how much influence the second brain wave feature has to diagnose mental diseases of the patient.

The weight of each second brain wave feature may be analyzed to have an influence on determination of the importance of the second brain wave feature. In addition, the importance of the second brain wave feature may be determined based on how many times the second brain wave feature has been used to diagnose mental diseases of the patient in the decision-making structure, a position of a stage where the second brain wave feature is used in the decision-making structure (whether the second brain wave feature is used in an initial branch stage, a middle branch stage, or a final branch stage), or the like.

Furthermore, according to an embodiment of the inventive concept, after diagnosing the mental diseases, in S460, the processor 230 may determine at least one brain region corresponding to the mental diseases among cerebral regions of the patient.

For example, the processor 230 may calculate a current source value of the brain cortex rather than a potential value of the head surface of the patient using a brain current source imaging algorithm. The brain current source imaging algorithm may uniformly divide the brain gray matter of the patient into voxels, each of which has a predetermined size, and may find a voxel having a significant correlation with the distribution of brain current sources and symptom severity of each voxel. In this case, the voxel having the significant correlation may be a brain region associated with the mental diseases among cerebral regions.

According to another embodiment of the inventive concept, the processor 230 may determine a brain region corresponding to mental diseases the patient is suffering from, using the machine learning model.

When determining the at least one brain region corresponding to the mental diseases the patient is suffering from among the cerebral regions, the processor 230 may be prescribed to stimulate the cerebral cortex corresponding to the determined brain region among cerebral cortices. Herein, the prescription may be generating stimulation information for stimulating at least one stimulation channel to stimulate a cerebral cortex of the at least one determined brain region.

In the inventive concept, the stimulation channel may be a channel associated with the cerebral cortex determined to need a stimulation signal by the processor 230 to treat mental diseases of the patient. Furthermore, the stimulation information may include at least one of at least one stimulation channel determined by the processor 230, a stimulation intensity of each stimulation channel, and a stimulation period of each stimulation channel.

According to an embodiment of the inventive concept, when the processor 230 generates stimulation information and provides the stimulation information to an external device (not shown) or a stimulation unit (not shown) of the XAI system 200, a medical team may perform treatment which stimulates a cerebral cortex depending on the information provided by the processor 230.

Referring to FIG. 5, the processor 230 may visualize and provide a diagnosed result 530 and a decision-making structure 510 which is description information for describing a basis for diagnosis.

As shown in FIG. 5, the processor 230 may compare any one of at least one second brain wave feature with a threshold in each stage of the decision-making structure 510. Furthermore, a next stage of comparing the second brain wave with the threshold may be determined as a result of the comparison. For example, the processor 230 may compare XAI-F1 which is one of second brain wave features with a first specific threshold in the highest stage of the decision-making structure 510. In this case, a second specific value to be compared in magnitude with XAI-F2 which is one of the second brain wave features in a second stage of the decision-making structure 510 may vary with the compared result.

In the inventive concept, second brain wave features compared in the comparison stage of the same order may be the same as or different from each other. Furthermore, thresholds compared with the respective second brain wave features may also be the same as or different from each other. For example, the second brain wave feature which is a comparison target in a second comparison stage of the decision-making structure 510 may be the same as XAI-F2. A threshold compared with XAI-F2 may vary with the compared result in an upper stage which is a first comparison stage. Furthermore, in a third comparison stage of the decision-making structure 510, the second brain wave feature which is a comparison target may be different as XAI-F3 or XAI-F5 and the threshold may be different as 70 or 50.

According to an embodiment of the inventive concept, the processor 230 may diagnose mental diseases of the patient based on the weight and the at least one second brain wave feature in the lowest stage of the decision-making structure 510. In detail, the processor 230 may assign the determined weight to the at least one second brain wave feature and may diagnose mental diseases of the patient by means of the machine learning model.

When completing the diagnosis of the mental diseases, the processor 230 may visualize and provide the diagnosed result 530 and the decision-making structure 510 used for diagnosis. Thus, the medical team may know the at least one second brain wave feature used for diagnosis of mental diseases of the patient, which is included in the decision-making structure 510, a threshold used as a comparison value, whether it is normal, and mental diseases the patient is suffering from, from the provided visual information. Furthermore, the processor 230 may provide information about a calculation formula used in the lowest stage together when diagnosing the mental diseases. The processor 230 may calculate a second brain wave feature and a weight using the calculation formula to diagnose mental diseases the patient is suffering from.

According to an embodiment of the inventive concept, the diagnosed result 530 may be information about at least one mental disease. Because most mental diseases are not independent of each other, the patient may be suffering from a plurality of mental diseases together. Thus, when the patient are suffering from the plurality of mental diseases as a result of diagnosing the mental diseases, the processor 230 may provide information about at least one mental diseases the patient is suffering from as a relative magnitude value.

When completing the diagnosis of the mental diseases, the processor 230 may visualize and provide the diagnosed result 530 and the decision-making structure 510 used for diagnosis. Thus, the medical team may identify information associated with a diagnostic basis of the XAI system 200 together with mental diseases the patient is suffering from, by means of the provided visual information, thus having reliability for the XAI system 200. Thus, because it is possible to explain the decision-making process leading to the diagnosis of mental illness to the medical team, the XAI system 200 according to an embodiment of the inventive concept may enhance reliability of a user for the diagnosed result, thus being actively used in the medical field.

According to an embodiment of the inventive concept, the processor 230 may provide the description information 520 for understanding the decision-making structure 510 together with the decision-making structure 510.

Herein, the description information 520 may include a description and importance for the at least one second brain wave feature. The description of the at least one second brain wave feature may include at least one of a channel name used to diagnose mental diseases of the patient, a brain wave type and brain wave power in the channel, and connectivity between channels. The importance of the at least one second brain wave feature is described above.

For example, referring to FIG. 5, the processor 230 may provide second brain wave features (XAI-F1, XAI-F2, . . . , XAI-Fm) used in diagnosing mental diseases of a specific patient, a name of a channel corresponding to each of the second brain wave features, a brain wave type and brain wave power in the used channel, and/or connectivity between channels together with importance for each of the second brain wave features, by means of the description information 520.

Furthermore, according to an embodiment of the inventive concept, the processor 230 may visualize and provide at least one stimulation channel which is a channel associated with a cerebral cortex of a cerebral region determined to need a stimulation signal to treat mental diseases of the patient.

Referring to FIG. 5, the processor 230 may provide visual information obtained by visualizing at least one stimulation channel to apply a stimulation signal through a channel 610. For example, as shown in FIG. 5, the processor 230 may display that a channel (Fp1, F1, FC4, P1, or PO3) 520 is a target needing stimulation to provide it as visual information to the medical team. Furthermore, the processor 230 may provide the medical team with information, including a stimulation intensity, a stimulation period, or the like necessary for the channel 520 as well as the channel 520, as visual information.

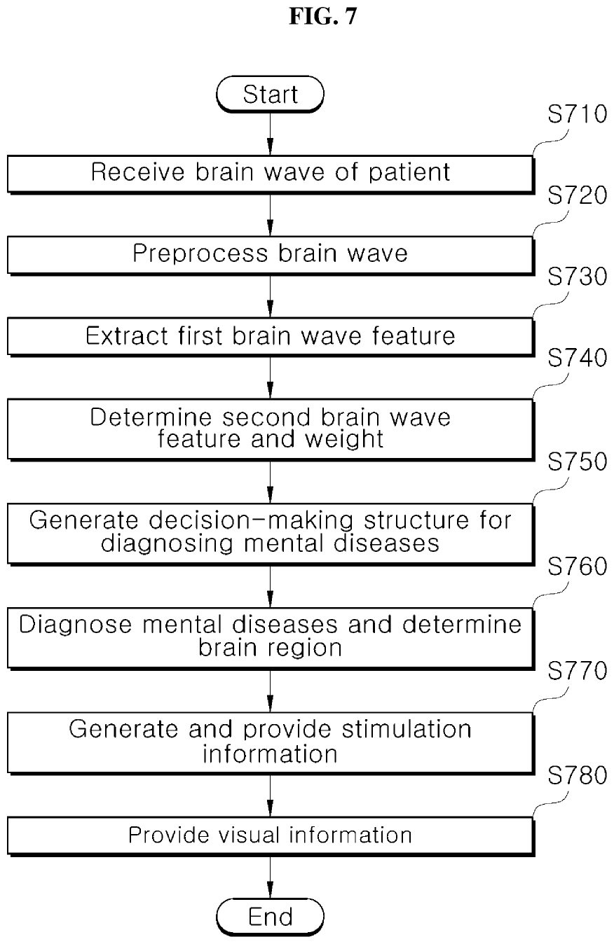

FIG. 7 is a flowchart illustrating diagnosing mental diseases and prescribing electroceuticals using an XAI system according to an embodiment of the inventive concept.

Respective operations of a method for diagnosing mental diseases according to the inventive concept may be performed by various types of electronic devices such as an XAI system 200 including a communication unit 210, a memory 220, and a processor 230.

Hereinafter, a description will be given in detail of an electroceutical prescription method according to the inventive concept by the processor 230 with reference to FIG. 7.

At least some or all of embodiments describing the XAI system 200 are applicable to the electroceutical prescription method. On the other hand, at least some or all of embodiments describing the method for diagnosing the mental diseases are applicable to the embodiments for the XAI system 200. Furthermore, an embodiment of the method for diagnosing the mental diseases according to disclosed embodiments is not limited to being performed by the XAI system 200 disclosed in the specification and may be performed by various types of electronic devices.

In S710, the XAI system 200 may receive a brain wave of a patient through the communication unit 210.

Next, in S720, the processor 230 may preprocess the received brain wave by means of noise cancellation and epoching processing.

According to an embodiment of the inventive concept, the preprocessing (S720) may fail to be performed by the processor 230. In detail, the preprocessing of the brain wave may be performed by means of an external device (not shown) or an external server (not shown). The XAI system 200 may receive only the preprocessed brain wave signal. In this case, the receiving (S710) of the brain wave of the patient and the preprocessing (S720) may be omitted.

Next, in S730, the processor 230 may extract at least one first brain wave feature from the preprocessed brain wave.

Next, in S740, the processor 230 may determine at least one second brain wave feature necessary to diagnose mental diseases of the patient and prescribe electroceuticals among the at least one first brain wave feature and a weight of the at least one second brain wave feature, using a machine learning model learned for diagnosis of mental diseases.

Next, in S750, the processor 230 may generate a decision-making structure for diagnosing mental diseases of the patient.

Next, in S760, the processor 230 may substitute the at least one second brain wave feature and the weight into the decision-making structure to diagnose mental diseases of the patient and may determine at least one brain region corresponding to the diagnosed result among cerebral regions of the patient.

Next, in S770, the processor 230 may generate stimulation information for stimulating at least one stimulation channel to stimulate a cerebral cortex of the at least one determined brain region and may provide the generated stimulation information.

In this case, the generated stimulation information may include at least one of at least one stimulation channel, a stimulation intensity, and a stimulation period. Furthermore, the generated stimulation information may be transmitted to an external device capable of stimulating a channel 620 of FIG. 6 (e.g., tES including tDCS or tACS, TMS, Visual Stimulus, or the like). A medical team may treat the patient depending to the transmitted stimulation information.

Alternatively, when the XAI system 200 includes a channel stimulation unit capable of applying an electrical signal (e.g., tES including tDCS or tACS, TMS, Visual Stimulus, or the like), the medical team may perform treatment which stimulates a cerebral cortex through the channel stimulation unit depending on the stimulation information generated by the processor 230.

Finally, in S780, the processor 230 may provide visual information obtained by visualizing the diagnosed result, the decision-making structure, and the at least one stimulation channel needing stimulation.

Herein, the visual information provided by visualizing them may be provided to an external display (not shown) having a display function through the communication unit 210. Alternatively, when including the display (not shown), the XIA system 200 may provide the medical team with the diagnosed result, a decision-making structure 510 of FIG. 5, description information 520 of FIG. 5, the channel 620, and the like by means of the display.

Various embodiments of the inventive concept may be implemented as software including one or more instructions stored in a storage medium (e.g., a memory) readable by a machine (e.g., the XAI system 200 or a computer). For example, a processor (e.g., the processor 230) of the device may invoke at least one of the stored one or more instructions from the storage medium, and execute it. This allows the machine to be operated to perform at least one function depending on the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium. For example, the “non-transitory storage medium” may include a buffer in which data is temporarily stored.

According to an embodiment, a method according to various embodiments disclosed in the specification may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smartphones) directly. If distributed online, at least part of the computer program product may be temporarily generated or at least temporarily stored in the machine-readable storage medium, such as memory of the manufacturer's server, a server of the application store, or a relay server. While the inventive concept has been described with reference to exemplary embodiments, it will be apparent to those skilled in the art that various changes and modifications may be made without departing from the spirit and scope of the inventive concept. Therefore, the embodiments described above are provided by way of example in all aspects, and should be construed not to be restrictive.

What is claimed is:

1. An explainable artificial intelligence system, comprising:
a communication unit configured to receive a brain wave of a patient; and
a processor configured to:
preprocess the received brain wave by means of noise cancellation and epoching processing;
extract at least one first brain wave feature from the preprocessed brain wave;
determine at least one second brain wave feature necessary to diagnose mental diseases of the patient among the at least one first brain wave feature and a weight of at least one second brain wave feature, using a machine learning model learned for diagnosis of mental diseases;
generate a decision-making structure for diagnosing the mental diseases of the patient, wherein an importance of the at least one second brain wave feature is determined in the process of generating the decision-making structure;
substitute the at least one second brain wave feature and the weight into the decision-making structure to diagnose the mental diseases of the patient; and
visualize and provide the diagnosed result, the decision-making structure which is description information for describing a basis for the diagnosis, a description of the at least one second brain wave feature, and the importance of the at least one second brain wave feature,
wherein the machine learning model uses a brain wave for each age and a brain wave feature for each channel, the brain wave feature being included in the brain wave for each age, as learning data to diagnose the mental diseases.

2. The explainable artificial intelligence system of claim 1, wherein the description of the at least one second brain wave feature includes a channel name necessary to diagnose the mental diseases of the patient, a brain wave type and brain wave power in the channel, and connectivity between channels, and
wherein the diagnosed result is information about at least one mental disease the patient is suffering from.

3. The explainable artificial intelligence system of claim 1, wherein the processor compares any one of the at least one second brain wave feature with a threshold, in each stage of the decision-making structure and determines a next comparison stage of comparing the second brain wave feature with the threshold, as a result of the comparison.

4. The explainable artificial intelligence system of claim 1, wherein the processor diagnoses the mental diseases of the patient based on the weight and the at least one second brain wave feature in the lowest stage of the decision-making structure.

5. The explainable artificial intelligence system of claim 1, wherein the machine learning model extracts the at least one second brain wave feature using an advanced variational autoencoder.

6. The explainable artificial intelligence system of claim 1, wherein the machine learning model is composed of an artificial neural network being in the form of an advanced variational autoencoder and is learned such that the at least one first brain wave feature is an input of the artificial neural network and such that the diagnosed result is output as an end result, and
wherein the artificial neural network is composed of a first recurrent neural network acting as an encoder and a second recurrent neural network acting as a decoder, and
wherein the input of the first recurrent neural network is the at least one first brain wave feature, the output of the first recurrent neural network is the at least one second brain wave feature, the input of the second recurrent neural network is the at least one second brain wave feature, and the output of the second recurrent neural network is the diagnosed result.

7. The explainable artificial intelligence system of claim 6, wherein a connection between the at least one first brain wave feature and a unit of the first recurrent neural network and a connection between the at least one second brain wave feature and a unit of the second recurrent neural network are all-to-all linear connections,
wherein a connection weight is randomly determined as uniform distribution, and
wherein a value of the connection weight is fixed in an initialization process and is then not changed.

8. The explainable artificial intelligence system of claim 7, wherein a connection between the unit of the first recurrent neural network and the at least one second brain wave feature and a connection between the unit of the second recurrent neural network and the diagnosed result are the all-to-all linear connections, and
wherein values of connection weights of the connection between the unit of the first recurrent neural network and the at least one second brain wave feature and the connection between the unit of the second recurrent neural network and the diagnosed result are changed while learned by a linear learning algorithm.

9. The explainable artificial intelligence system of claim 8, wherein the values of the connection weights of the connection between the unit of the first recurrent neural network and the at least one second brain wave feature and the connection between the unit of the second recurrent neural network and the diagnosed result are randomly determined as uniform distribution and are then changed while learned by the linear learning algorithm.

10. The explainable artificial intelligence system of claim 9, wherein the generating of the decision-making structure includes:
representing a result learned from the first recurrent neural network to the at least one second brain wave feature, the second recurrent neural network, and the diagnosed result as a first formula in the machine learning model; and
converting the represented first formula into the decision-making structure.

11. The explainable artificial intelligence system of claim 10, wherein the importance of the at least one second brain wave feature is determined in the process of converting the decision-making structure, and
wherein the importance of the at least one second brain wave feature is obtained by digitizing an influence of the second brain wave feature to diagnose the mental diseases of the patient.

12. The explainable artificial intelligence system of claim 7, wherein the first recurrent neural network is composed of a plurality of units,
wherein the number of the units making up the first recurrent neural network is determined to be greater than 100 times the number of the at least one first brain wave feature.

13. The explainable artificial intelligence system of claim 12, wherein the units making up the first recurrent neural network are randomly and recurrently connected with each other and a connection probability between the respective units is determined between from 0.1% to 1%,
wherein a connection weight between the units making up the first recurrent neural network is determined as uniform distribution among values between from −1 to 1 and a certain scaling factor is multiplied by connection weights such that an absolute value of the largest eigen value of a connection weight matrix determined subsequently becomes 1 or less, and
wherein the calculated connection weight matrix is subsequently fixed and is not changed.

14. The explainable artificial intelligence system of claim 1, wherein the learning data further includes feedback information of a medical team about the diagnosis of the mental diseases.

15. The explainable artificial intelligence system of claim 1, wherein the processor determines at least one brain region corresponding to the diagnosed result among cerebral regions of the patient, generates stimulation information for stimulating at least one stimulation channel to stimulate a cerebral cortex of the at least one determined brain region and provides the generated stimulation information, and further visualizes and provides the at least one stimulation channel other than the diagnosed result, the decision-making structure, the description of the at least one second brain wave feature, and the importance of the at least one second brain wave feature, and
wherein the machine learning model further uses a treatment progress according to stimulation for each region of the cerebrum as learning data other than the brain wave for each age and the brain wave feature for each channel, the brain wave feature being included in the brain wave for each age, to diagnose the mental diseases.

16. A control method of an explainable artificial intelligence system, the control method comprising:
receiving a brain wave of a patient;
preprocessing the received brain wave by means of noise cancellation and epoching processing;
extracting at least one first brain wave feature from the preprocessed brain wave;
determining at least one second brain wave feature necessary to diagnose mental diseases of the patient among the at least one first brain wave feature and a weight of the at least one second brain wave feature, using a machine learning model learned for diagnosis of mental diseases;
generating a decision-making structure for diagnosing the mental diseases of the patient, wherein an importance of the at least one second brain wave feature is determined in the process of generating the decision-making structure;
substituting the at least one second brain wave feature and the weight into the decision-making structure to diagnose the mental diseases of the patient; and
visualizing and providing the diagnosed result, the decision-making structure which is description information for describing a basis for the diagnosis, a description of the at least one second brain wave feature, and the importance of the at least one second brain wave feature, and
wherein the machine learning model uses a brain wave for each age and a brain wave feature for each channel, the brain wave feature being included in the brain wave for each age, as learning data to diagnose the mental diseases.

17. A computer-readable storage medium storing a program for implementing the control method of the explainable artificial intelligence system of claim 16.
